(19) United States
(12) Patent Application Publication
(10) Pub. No.: US 2024/0068858 A1
(43) Pub. Date: Feb. 29, 2024

(54) Apparatus and method for treating containers having a sensor device for fill level measurement

(71) Applicant: KRONES AG, Neutraubling (DE)

(72) Inventors: Juergen SOELLNER, Beratzhausen (DE); Daniel THEEN, Lappersdorf (DE); Florian GERNGROSS, Regensburg (DE); Christian TRAUNER, Nittenau (DE)

(21) Appl. No.: 18/239,667

(22) Filed: Aug. 29, 2023

(30) Foreign Application Priority Data
Aug. 29, 2022 (DE) 10 2022 121 827.9

(51) Int. Cl. G01F 23/00

(52) U.S. Cl. CPC G01F 23/0053

(57) ABSTRACT

Disclosed is an apparatus for treating containers with a transport device which transports the containers to be treated along a predetermined transport path, wherein said transport device has a rotatable transport carrier at which a plurality of treatment stations for treating the containers is arranged, wherein the apparatus has a clean room within which the containers are treated in a predetermined manner, and with a sealing device which seals said clean room from an environment, wherein said sealing device has a reservoir which can be filled with a liquid medium, and with a sensor device for measuring a filling level of said liquid medium at least indirectly. The sensor device is arranged in a supply line which can be brought into flow connection with the reservoir.

### BACKGROUND OF THE INVENTION

The present invention relates to an apparatus and a method for treating containers and in particular plastic preforms and/or plastic containers. Such apparatuses and methods have long been known from the prior art. During such a treatment, the plastic preforms are formed, for example, into plastic containers or filled with a flowable medium, such as a beverage, within a filling device.

During forming, the plastic preforms are usually formed into the plastic containers within a forming station and more precisely within a blow molding device by applying a flowable medium. The blow molding devices usually have two blow mold side parts and a bottom part, which together form a hollow space which corresponds to a negative contour of the finished plastic container. So-called blow molding machines are also known from the prior art which, during the blowing process, also stretch the plastic preforms in their longitudinal direction. These are in particular so-called stretch blow molding machines.

In recent times, apparatuses and methods have also become known with which the formation of the plastic preforms into the plastic containers takes place within a clean room. In this way, it is, for example, possible for the entire forming process to be carried out under sterile conditions, and the manufactured plastic containers therefore do not have to be sterilized again or only with less effort.

A plurality of forming stations is arranged on a movable and, in particular, rotatable transport carrier, which is arranged within the clean room. In this case, the transport carrier is sealed from a stationary part of the apparatus and a non-sterile environment by means of a sealing device, such as, in particular, a water lock.

It is known from the internal prior art of the applicant that the sealing device has a receiving space filled with a liquid, wherein a circumferential rotating wall projects into the liquid. In order to monitor the filling level of the receiving space, a sensor device is arranged directly on the sealing device. In this case, the sensor device serves to indicate, in particular, a too low a filling level of the liquid since the sealing effect can no longer be ensured if the filling level is too low.

If the receiving space contains too much liquid, for example due to condensation and the like, the receiving space overflows. Even too high circumferential speeds of the rotatable transport carrier can cause liquid to spill out of the receiving space since an interference contour arises on the outer ring. In both cases, the (possibly contaminated) liquid from the receiving space enters the clean room in an uncontrolled manner.

The object of the present invention is therefore to provide an apparatus and a method with which the contamination of the clean room with the sealing liquid can be prevented.

### SUMMARY OF THE INVENTION

The invention is therefore directed to an apparatus for treating containers, preferably plastic preforms and/or plastic containers, with a transport device which transports the containers to be treated along a predetermined transport path, wherein said transport device has a rotatable transport carrier at which a plurality of treatment stations for treating the containers is arranged, wherein the apparatus has a clean room within which the containers are treated in a predetermined manner, and with a sealing device which seals said clean room from an environment, wherein said sealing device has a reservoir which can be filled with a liquid medium, and with a sensor device for measuring a filling level of said liquid medium at least indirectly.

According to the invention, the sensor device is arranged in a supply line which can be brought into flow connection with the reservoir. Preferably, the sensor device is arranged on an overflow container which can also be brought into flow connection with the supply line.

As a result of this arrangement, the aforementioned interference contour on the outer side of the sealing device or of the water lock tank is avoided so that a spillover through this interference contour is precluded. The filling level of the liquid within the overflow container is relatively constant and is in particular not influenced by the rotation of the transport carrier. In the event of an increase in the liquid level within the overflow container, the liquid can additionally be discharged in particular in a targeted manner from said overflow container and thus no longer flows in an uncontrolled manner into the clean room.

In a preferred embodiment, the supply line is a supply line for the liquid medium, wherein the supply line is in flow connection with the reservoir and the overflow container. If the filling level of the flowable medium within the reservoir rises, a portion of the liquid medium can flow out into the overflow container via the supply line and can be discharged in a targeted manner from there. This prevents the liquid medium from spilling out of the reservoir.

In a further preferred embodiment, a portion of the liquid medium is therefore discharged from the overflow container via an overflow device if a predetermined filling level is exceeded. Particularly preferably, the liquid medium is guided in a targeted manner in a predetermined direction or into a predetermined region. In particular, this prevents the liquid medium from entering the clean room in an uncontrolled manner. It would also be conceivable for the liquid medium to be discharged via the supply line.

The apparatus for treating containers is preferably an apparatus for forming plastic preforms into plastic containers, a filling device for filling the containers, a closing device for closing the containers, a sterilization device for sterilizing the containers, or the like.

The apparatus for forming plastic preforms into plastic containers is preferably a blow molding machine. In an advantageous embodiment, a plurality of forming stations is arranged on a common movable transport carrier. This carrier is in particular a rotatable carrier and in particular a blowing wheel.

In this case, the forming stations each have blow molding devices which are formed in multiple parts and have two blow mold side parts and a bottom part. These blow mold side parts are preferably detachably arranged on a mold carrier shell or on the blow mold carriers. The blow mold carriers are pivotable relative to one another in order to open and close the blow molding devices. In addition, the blow mold carrier has locking mechanisms in order to lock the mold halves against each other during the blowing process.

Advantageously, a plurality of such blow molding devices is arranged within a blow molding machine and particularly preferably within a stretch blow molding machine. This means that the plastic preforms are expanded into the plastic containers by applying compressed air. For this purpose, the apparatus preferably has a blowing nozzle which can be applied to an opening of the plastic preforms in order to apply blowing air to the plastic preforms and expand them by means of blowing air. In addition, a valve arrangement is preferably also provided which controls the supply of the blowing air to the plastic preforms.

In a further advantageous embodiment, the blow molding devices or the forming stations each have stretching rods which stretch the plastic preforms in their longitudinal direction. Particularly preferably, the blow molding machine is, or the carriers and the forming stations are, arranged within a clean room which delimits the blow molding machine from a non-sterile environment. Drive devices for closing, locking and/or opening the blow molds are preferably arranged outside the cleanroom.

The forming stations are preferably transported within the clean room, wherein the clean room is preferably bounded by a plurality of walls. Preferably, the clean room is bounded by at least one standing wall and a wall that moves relative to this standing wall. In this case, for example, the transport carrier at which the forming stations and/or blow molding devices are arranged can already have or form one of these walls, and in particular the moving wall. The clean room delimits the forming stations and/or blow molding devices in particular from a non-sterile environment.

In a further preferred method, parts of the blow molding device are tempered. In particular, parts of the blow molding device are heated. This heating is preferably carried out by means of electrical energy or by means of a flowable and in particular liquid temperature control medium. For example, hot oil or water can be used to temper the side parts of the blow mold and/or the bottom part of the blow mold. This tempering can be carried out by means of channels which are arranged in the blow mold itself, and/or also by means of channels which are arranged in blow mold shells and/or also in a blow mold carrier.

In a preferred embodiment, the reservoir is designed in the form of a circumferential channel. This circumferential channel of the reservoir can be filled with a liquid medium, such as in particular water, via the supply line.

In a further preferred embodiment, the sealing device has a circumferential wall which projects into the circumferential channel, wherein this circumferential wall is preferably designed to be rotatable. The sealing device preferably seals the clean room from a non-sterile environment. In particular, the sealing device prevents non-sterile ambient air and germs from entering and contaminating the clean room.

The circumferential wall, which projects into the circumferential channel, is preferably at least one blade which forms a component of the water lock or of the hydraulic seal. Particularly preferably, this wall dips into the liquid located in the channel. This blade usually belongs to a rotating part of the system and the circumferential channel is arranged in a stationary manner. The circumferential wall of the sealing device is therefore preferably designed to be rotatable with respect to the channel. Preferably, the rotational movement of this wall is coupled to the rotational movement of the transport carrier.

The circumferential channel is preferably divided by the circumferential wall of the sealing device into a radially inner channel section and a radially outer channel section, wherein the blade preferably dips into the liquid in a region between the inner channel section and the outer channel section.

The sensor device is preferably in contact with the liquid medium within the overflow container. In a preferred embodiment, the sensor device outputs a signal if the sensor device is no longer in contact with the liquid medium. The sensor device therefore preferably indicates if the filling level of the liquid medium is too low, since sealing cannot be ensured if the filling level is too low.

In a preferred embodiment, the circumferential channel is preferably automatically filled with liquid medium again via the supply line as soon as the sensor device detects that the filling level is too low.

In a further preferred embodiment, the liquid medium is discharged from the overflow container via an overflow device if a predetermined filling level is exceeded. As already mentioned above, the liquid medium is directed to a region of the apparatus that is located outside the clean room, so that the latter is not contaminated with germs by the liquid medium. The sensor device and the overflow container are therefore particularly preferably arranged outside the clean room.

The present invention is further direct to a method for treating containers, preferably plastic preforms and/or plastic containers, wherein a transport device transports the containers to be treated along a predetermined transport path, and said transport device has a rotatable transport carrier at which a plurality of treatment stations for treating the containers is arranged, with a clean room within which the containers are treated in a predetermined manner, and with a sealing device which seals said clean room from an environment, wherein said sealing device has a reservoir filled with a liquid medium, and a sensor device determines a filling level of said liquid medium in said reservoir at least at times.

According to the invention, the sensor device is arranged in a supply line which is in flow connection with the reservoir at least at times.

In this case, the above-described apparatus is in particular designed and provided to carry out this described method, i.e., all listed features of the above-described apparatus are also disclosed for the method described here and vice versa.

Further advantages and embodiments emerge from the accompanying drawing.

### BRIEF DESCRIPTION OF THE DRAWINGS

In the drawings:

FIG. 1 shows a schematic representation of an apparatus for treating containers;

FIG. 2 shows a schematic representation of the sensor device according to the invention; and

FIG. 3 shows a schematic representation of the sealing device.

### DETAILED DESCRIPTION OF THE INVENTION

FIG. 1 shows an apparatus 1 for treating containers and in particular plastic containers and/or plastic preforms. In this apparatus 1, the plastic preforms are fed to the apparatus 1 via a supply device 62 and, after forming, the plastic containers 15 are discharged from the apparatus 1 via a discharge device 64. This apparatus is therefore an apparatus 1 for forming plastic preforms into plastic containers 15.

The apparatus 1 has a rotatable carrier 22 at which a plurality of treatment stations 4, in particular forming stations, are arranged. These individual treatment or forming stations each have blow molding devices 82, which in their interior form a cavity for expanding the plastic preforms.

Reference sign 84 denotes an application device, which is used to expand the plastic preforms 10. Reference sign 88 denotes a stretching rod used to stretch the plastic preforms in their longitudinal direction. Preferably, all forming stations have such blow molds 82 and stretching rods 88. The flowable medium is guided to the individual treatment stations via at least one annular channel 2a via lines (not shown).

Reference sign 8 schematically denotes a clean room, which is preferably annular here and surrounds the transport path of the plastic preforms 10. Preferably, a (geometric) axis of rotation, with respect to which the transport carrier is rotatable, is arranged outside the clean room 8. Preferably, the clean room is sealed from the non-sterile environment by a sealing device, which preferably has at least two water locks.

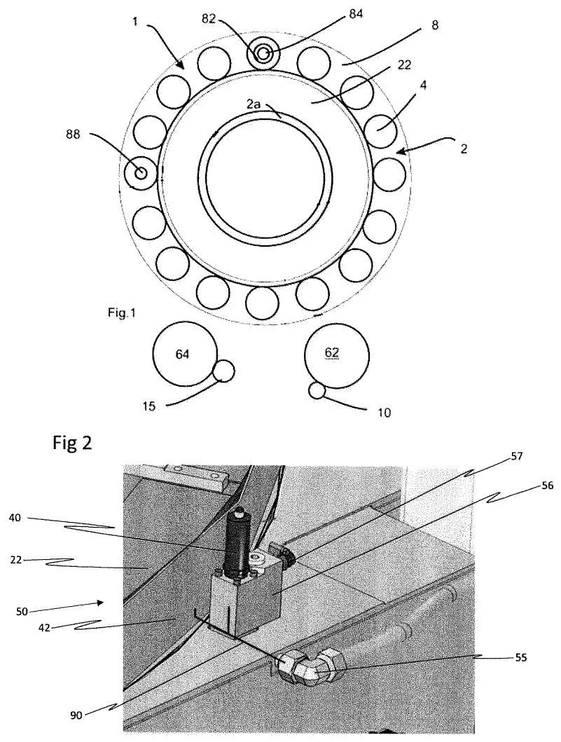

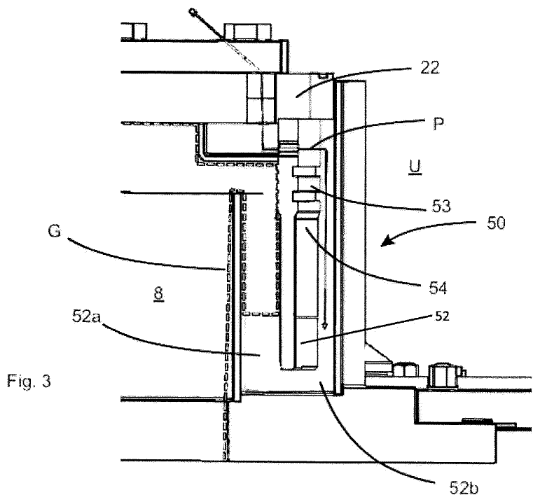

FIG. 2 is a schematic representation of a sensor device 40 according to the invention. The sensor device 40 is arranged on an overflow container 56 with an overflow device 57, via which the liquid medium can be discharged.

The reservoir 42 for the liquid medium and the overflow container 56 and/or the sensor device 40 are in this case in flow connection 90 with a supply line 55 for the liquid medium. Reference sign 50 denotes the sealing device which seals the rotatable transport carrier 22 from a stationary part of the apparatus or delimits the clean room, in which the transport carrier 22 is arranged, from the environment.

FIG. 3 shows a detailed view of the sealing device 50. In this case, a circumferential channel 52 is shown, in which a liquid, such as water (not shown), is located. In this case, said circumferential channel 52 is stationary or belongs to the stationary part of the apparatus.

A circumferential wall 54 or a blade dips into the channel 52. Said circumferential wall 54 is preferably designed to be rotatable or forms a component of the rotating part of the apparatus. The circumferential wall 54 is preferably arranged on the transport carrier 22.

Said circumferential wall 54 divides the channel 52 into a first or inner sub-channel 52a and an outer or second sub-channel 52b. In this case, the inner sub-channel 52a preferably also forms a region of the clean room 8, while the outer sub-channel 52b forms a region of the (non-sterile) environment U. The dashed line G denotes the boundary of the clean room 8.

The applicant reserves the right to claim all features disclosed in the application documents as essential to the invention, provided that they are novel over the prior art individually or in combination. It is also pointed out that features which can be advantageous in themselves are also described in the individual figures. The person skilled in the art will immediately recognize that a particular feature described in a figure can be advantageous even without the adoption of further features from this figure. Furthermore, the person skilled in the art will recognize that advantages can also result from a combination of several features shown in individual or in different figures.

### LIST OF REFERENCE SIGNS

1 apparatus
2 transport device
2a annular channel
4 treatment stations
8 clean room
10 plastic preform
15 container
22 transport carrier
40 sensor device
42 reservoir
50 sealing device
52 circumferential channel
52a inner sub-channel
52b outer sub-channel
54 circumferential wall
55 supply line
56 overflow container
57 overflow device
62 supply device
64 discharge device
82 blow molding device
84 application device
88 stretching rod
90 flow connection
U environment
G boundary of the clean room 8

1. An apparatus for treating containers with a transport device which transports the containers to be treated along a predetermined transport path, wherein said transport device has a rotatable transport carrier at which a plurality of treatment stations for treating the containers is arranged, wherein the apparatus has a clean room within which the containers are treated in a predetermined manner, and with a sealing device which seals said clean room from an environment, wherein said sealing device has a reservoir which can be filled with a liquid medium, and with a sensor device for measuring a filling level of said liquid medium at least indirectly,
wherein
the sensor device is arranged in a supply line which can be brought into flow connection with the reservoir.

2. The apparatus according to claim 1,
wherein
the sensor device is arranged on an overflow container.

3. The apparatus according to claim 1,
wherein
the supply line is a supply line for the liquid medium, wherein the supply line is in flow connection with the reservoir and the overflow container.

4. The apparatus according to claim 2,
wherein
a portion of the liquid medium is discharged from the overflow container via an overflow device if a predetermined filling level is exceeded.

5. The apparatus according to claim 1,
wherein
the reservoir is designed in the form of a circumferential channel.

6. The apparatus according to claim 5,
wherein
the sealing device has a circumferential wall which projects into the circumferential channel, wherein said circumferential wall is preferably designed to be rotatable.

7. The apparatus according to claim 4,
wherein
the sensor device outputs a signal if the sensor device is no longer in contact with the liquid medium.

8. The apparatus according to claim 7,
wherein
the circumferential channel is automatically filled with liquid medium again as soon as the sensor device detects that the filling level is too low.

9. The apparatus according to claim 2,
wherein
the sensor device and the overflow container are arranged outside the clean room.

10. A method for treating containers, wherein a transport device transports the containers to be treated along a predetermined transport path, and said transport device has a rotatable transport carrier at which a plurality of treatment stations for treating the containers is arranged, with a clean room within which the containers are treated in a predetermined manner, and with a sealing device which seals said clean room from an environment, wherein said sealing device has a reservoir filled with a liquid medium, and a sensor device determines a filling level of said liquid medium in said reservoir at least intermittently,
wherein
the sensor device is arranged in a supply line which is at least at times in flow connection with the reservoir.
